(19) United States
(12) Patent Application Publication
(10) Pub. No.: US 2025/0140336 A1
(43) Pub. Date: May 1, 2025

(54) SYSTEM AND METHOD FOR DISCOVERING CANDIDATE MATERIALS FOR INFECTIOUS DISEASE TREATMENT

(71) Applicants: AIcloud Co., Ltd., Pohang-si (KR); POSTECH RESEARCH AND BUSINESS DEVELOPMENT FOUNDATION, Pohang-si (KR)

(72) Inventors: Toohyon CHO, Pohang-si (KR); Chan Kyu PARK, Pohang-si (KR); Dongwoo KIM, Pohang-si (KR); Jaeseung HEO, Jinju-si (KR); Jungwoo SON, Pohang-si (KR); Hwanjo YU, Pohang-si (KR)

(21) Appl. No.: 18/687,883

(22) PCT Filed: Jun. 10, 2022
(86) PCT No.: PCT/KR2022/008217

§ 371 Date: Feb. 29, 2024

(30) Foreign Application Priority Data
Sep. 2, 2021 (KR) 10-2021-0117238
Apr. 29, 2022 (KR) 10-2022-0053736

(51) Int. Cl. G16B 15/30; G06N 3/042; G06N 3/045; G16B 40/20

(52) U.S. Cl. CPC G16B 15/30; G06N 3/042; G06N 3/045; G16B 40/20

(57) ABSTRACT

The present disclosure relates to a system and method for discovering candidate materials for treatment.

According to an embodiment, the system for discovering candidate materials for treatment includes a prediction system that inputs first graph data of a target protein and second graph data of the candidate materials to a prediction model and determines whether the candidate materials are candidate materials for treatment of the target protein based on an output value output from the prediction model in response to the first and second graph data, in which the prediction model may be a graph neural networks (GNN)-based model for predicting presence or absence of binding between the target protein and the candidate material.

### TECHNICAL FIELD

The present disclosure relates to a system and method for discovering candidate materials for treatment, and more particularly, to a system and method for discovering candidate materials for infectious disease treatment.

### BACKGROUND ART

Typically, developing a new drug requires a lot of cost and a long development period. For example, the development of a compound new drug takes approximately 10 to 12 years and a cost of $1 billion, and the development of a bio new drug takes approximately 4 to 5 years and a cost of $500 million. Therefore, in a situation where an infectious disease such as the recently emerged COVID-19 spreads worldwide, the development of treatment is delayed, which may become a serious problem worldwide.

The development of new drugs is mainly accomplished by extracting plants, animals, microorganisms, and marine organisms from the natural world or creating synthetic derivatives. However, in recent years, materials extracted from the natural world are gradually being depleted, and therefore, the development of new drugs using the materials extracted from the natural world is also gradually decreasing.

### DISCLOSURE

### Technical Problem

The present disclosure attempts to provide a system and method for discovering candidate materials for treatment capable of shortening time required to develop new drugs by supporting the discovery of candidate materials for infectious disease treatment.

### Technical Solution

According to an embodiment, a system for discovering candidate materials for treatment may include a prediction system that inputs first graph data of a target protein and second graph data of the candidate materials to a prediction model and determines whether the candidate materials are candidate materials for treatment of the target protein based on an output value output from the prediction model in response to the first and second graph data. The prediction model may be a graph neural networks (GNN)-based model for predicting presence or absence of binding between the target protein and the candidate material.

The prediction model may include: a first multi-layer graph isomorphism network (GIN) that embeds input graph data into a first vector; a second multi-layer GIN that embeds the input graph data into a second vector; and a classifier that generates the output value by passing a third vector generated by combining the first vector and the second vector through a multi-layer perceptron (MLP). The first and second graph data may be input to the first and second multi-layer GINs, respectively.

The first and second multi-layer GINs may each be composed of a 5-layer GIN.

The first vector and the second vector may each be a 32-dimensional vector, and the third vector may be a 64-dimensional vector.

The MLP may be a two-layer MLP.

The prediction system may convert feature data of the target protein and the candidate materials into the first graph data and the second graph data, respectively, and the feature data may be amino acid sequence data for a binding site.

The system may further include a learning system that trains the prediction model using a plurality of training data sets. The plurality of training data sets may include a plurality of target proteins and feature data for antibodies of each of the plurality of target proteins. The learning system may convert feature data of the target protein and antibody corresponding to each other into third graph data and fourth graph data, respectively, and train the prediction model using the third graph data and the fourth graph data.

The learning system may calculate a loss using the output value output from the prediction model and a loss function after the third graph data and the fourth graph data are input to the prediction model, and train the prediction model in a direction in which the loss is minimized.

A method for discovering candidate materials for treatment in a system for discovering candidate materials for treatment may include: inputting first graph data of a target protein and second graph data of the candidate materials to a prediction model to obtain a prediction value for presence or absence of binding between the target protein and the candidate material; and determining whether the candidate materials are candidate materials for treatment of the target protein based on the prediction value. The prediction model may be a GNN-based model for predicting presence or absence of binding between the target protein and the candidate material.

The acquiring of the prediction value may include: embedding the first and second graph data into first and second vectors, respectively, through first and second multi-layer GINs constituting the prediction model; and acquiring the prediction value by passing a third vector generated by combining the first and second vectors through an MLP of a classifier constituting the prediction model.

The first and second multi-layer GINs may each be composed of a 5-layer GIN, and the first vector and the second vector may each be a 32-dimensional vector and the third vector may be a 64-dimensional vector.

The method may further include converting feature data of the target protein and the candidate materials into the first graph data and the second graph data, respectively, in which the feature data may be amino acid sequence data for a binding site.

The method may further include: converting feature data of the target protein and antibody corresponding to each other into third graph data and fourth graph data, respectively; and training the prediction model using the third graph data and the fourth graph data.

The training may include: embedding the third and fourth graph data into fourth and fifth vectors, respectively, through first and second multi-layer GINs constituting the prediction model; acquiring a prediction value by passing a sixth vector generated by combining the fourth and fifth vectors through the MLP of the classifier constituting the prediction model; calculating a loss using the prediction value output from the prediction model and a loss function; and training the prediction model in a direction in which the loss is minimized.

The prediction value output from the prediction model may include a classification prediction value for the presence or absence of binding between the target protein and antibody corresponding to the third and fourth graph data, and the training of the prediction model in the direction in which the loss is minimized may include training the prediction model in a direction in which a binary cross-entropy loss between the classification prediction value and actual data decreases using an Adam optimizer.

The prediction value output from the prediction model may include a regression prediction value for binding force of the target protein and antibody corresponding to the third and fourth graph data, and the training of the prediction model in the direction in which the loss is minimized may include training the prediction model in a direction in which a mean squared error loss between the regression prediction value and actual data decreases using an Adam optimizer.

### Advantageous Effects

According to the present disclosure, it is possible to provide a discovery system with improved performance and accuracy in discovering candidate materials for infectious disease treatment.

### DESCRIPTION OF THE DRAWINGS

FIG. 1 schematically illustrates a system for discovering candidate materials for treatment according to an embodiment.

FIG. 2 is a diagram schematically illustrating a structure of a discovery model according to an embodiment.

FIG. 3 is a diagram schematically illustrating a method for generating a discovery model of a learning system according to an embodiment.

FIG. 4 is a diagram schematically illustrating a method for discovering candidate materials of a prediction system according to an embodiment.

### MODE FOR INVENTION

Hereafter, embodiments of the present disclosure will be described in detail with reference to the accompanying drawings and the same or similar components are given the same reference numerals and are not repeatedly described. The suffix “module” and/or “unit” for components used in the following description is given or mixed in consideration of only the ease of writing of the specification, and therefore, do not have meanings or roles that distinguish from each other in themselves. Further, when it is decided that a detailed description for the known art related to the present disclosure may obscure the gist of the present disclosure, the detailed description will be omitted. Further, it should be understood that the accompanying drawings are provided only in order to allow exemplary embodiments of the present disclosure to be easily understood, and the spirit of the present disclosure is not limited by the accompanying drawings, but includes all the modifications, equivalents, and substitutions included in the spirit and the scope of the present disclosure.

Terms including an ordinal number such as first, second, etc., in this disclosure may be used to describe various components, but the components are not limited to these terms. The above terms are used solely for the purpose of distinguishing one component from another.

Singular forms are to include plural forms unless the context clearly indicates otherwise.

It will be further understood that terms “include” or “have” used in the present specification specify the presence of features, numerals, steps, operations, components, parts mentioned in the present specification, or combinations thereof, but do not preclude the presence or addition of one or more other features, numerals, steps, operations, components, parts, or combinations thereof.

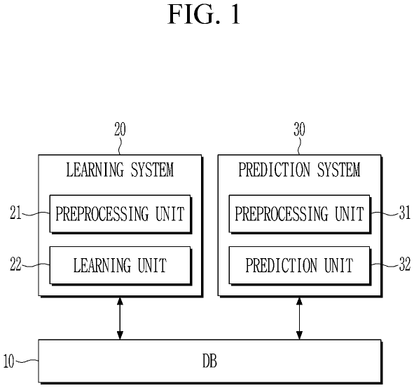

FIG. 1 schematically illustrates a system for discovering candidate materials for treatment according to an embodiment.

Referring to FIG. 1, a system 1 for discovering candidate materials for treatment according to an embodiment may include a database (DB) 10, a learning system 20, and a prediction system 30.

The database 10 may store data used or processed in the system 1 for discovering candidate materials for treatment.

The database 10 may store a discovery model for discovering candidate materials for infectious disease treatment. The discovery model is an artificial neural network-based prediction model that inputs graph data of a target protein for discovery of treatment and the corresponding candidate materials for treatment, and outputs a prediction value for binding possibility between the target protein and the candidate material.

The database 10 may store training data sets used for training the discovery model. Each training data set may include feature data (for example, amino acid sequence data for a binding site) of a pre-verified target protein and the corresponding antibody, respectively.

The database 10 may store the target protein for discovery of the candidate materials for treatment and feature data (for example, amino acid sequence data for binding sites) of the corresponding candidate materials for treatment.

The learning system 20 may perform artificial intelligence-based training using the training data sets stored in the database 10 to generate a discovery model for discovering candidate materials for infectious disease treatment.

The learning system 20 may include a preprocessing unit 21 and a learning unit 22.

The preprocessing unit 21 may perform a preprocessing process to convert the training data set read from the database 10, that is, the feature data of the antibody and target protein, respectively, into graph data (G=(X, A)). Here, X may denote a node feature matrix representing the information of vertices of the graph, and A may denote an adjacency matrix representing a connection of each node.

The learning unit 22 may learn a graph neural networks (GNN)-based discovery model using the training data sets converted into the graph data through the preprocessing process. The GNN is an artificial neural network that uses the graph data as input. The GNN may receive graph data including a graph structure (connection relationships between nodes) and feature information for each node, and output embedding for each node based on the information on the input features and information on neighboring nodes.

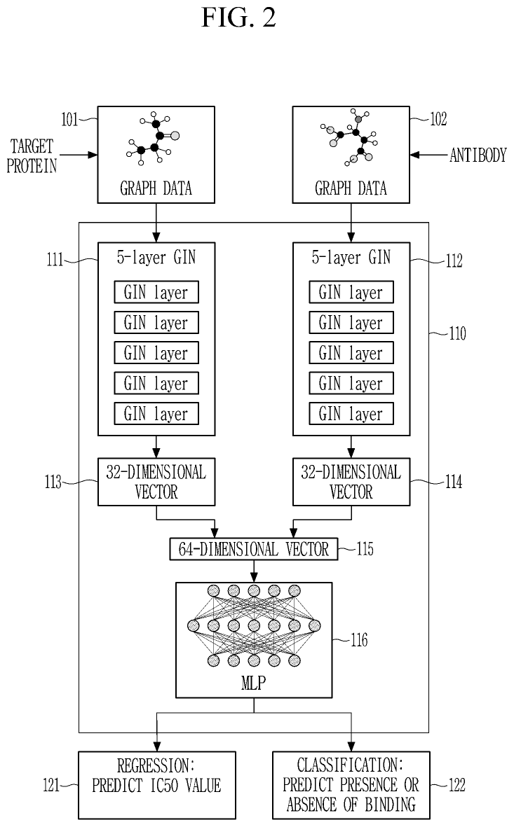

FIG. 2 is a diagram schematically illustrating a structure of a discovery model according to an embodiment.

Referring to FIG. 2, the discovery model 110 may include two 5-layer graph isomorphism networks (GINs) 111 and 112, and a classifier 116.

The GIN is a network that improves expressiveness of structural characteristics of the graph in the GNN by using injective aggregation functions and sum pooling readout. The GIN uses a method of comparing performance of a 1-Weisfeiler-Lehman (WL) test in a graph isomorphism problem to indicate the performance of the GNN. The graph isomorphism problem is a problem of determining whether two graphs have the same topological structure. Simply, the graph isomorphism problem may be said to be a problem of comparing the structures of two graphs. The 1-WL test is an algorithm designed to solve the graph isomorphism problem, which takes a long time to solve, in a short time even if the accuracy is low. The 1-WL test uses a method of continuously updating node features using adjacent node features and a hash function, and has the advantage of being able to distinguish most graphs in a short time.

In the GNN, as the number of layers increases, the node features have similar values, which may cause an over-smoothing problem that drastically reduces performance. Therefore, when the GIN is configured to have too many layers, the training performance may rather deteriorate. Accordingly, as illustrated in FIG. 2, the discovery model 110 may be configured using 5-layer GINs 111 and 112 composed of 5 GIN layers.

Referring to FIG. 2, each 5-layer GIN 111 and 112 may perform graph embedding to convert the input graph data 101 and 102 into 32-dimensional vectors 113 and 114, respectively. The graph embedding means converting the graph into a vector or set of vectors. The 5-layer GIN 111 may embed the graph data 101 corresponding to the target protein into the 32-dimensional vector 113, and the 5-layer GIN 112 may embed the graph data 102 corresponding to the antibody into the 32-dimensional vector 114.

The 32-dimensional vectors 113 and 114 output from the 5-layer GINs 111 and 112 become a 64-dimensional vector 115 through simple combination (concatenation), and the 64-dimensional vector (115) thus obtained may be input to the classifier 116 of the discovery model 110.

The classifier 116 may be composed of a 2-layer multi-layer perceptron (MLP). The classifier 116 may generate a one-dimensional output value by passing the input 64-dimensional vector through the 2-layer MLP. For example, as illustrated in FIG. 2, the classifier 116 may output a regression prediction value 121 and a classification prediction value 122 as output values.

The classifier 116 may adjust the output value of the 2-layer MLP to a value between 0 and 1 using a sigmoid function, and then predict that the antibody binds to the target protein used as input when the output value is greater than or equal to a predetermined value (for example, 0.5), or otherwise, predict that the corresponding target protein does not bind to the antibody. In addition, the classifier 116 may generate a classification prediction value 122 based on the prediction result for the presence or absence of such binding and output the generated classification prediction value 122.

The classifier 116 may use the output value of the 2-layer MLP as an inhibitory concentration 50 (IC50) value to output the regression prediction value 121 corresponding to binding force between the target protein and the antibody used as input.

Referring back to FIG. 1, when the output values (regression prediction value 121 and classification prediction value 122) are output from the discovery model 110, the learning unit 22 may calculate a loss by substituting the output values (regression prediction value 121 and classification prediction value 122) and data label into a criterion of a loss function. In addition, the learning unit 22 may train the discovery model 110 in a direction that the loss calculated by the loss function through optimization is minimized.

For example, the learning unit 22 may use an Adam optimizer to train the discovery model 110 in a direction in which a binary cross-entropy loss between the classification prediction value 122 output from the discovery model 110 and actual data decreases. In addition, for example, the learning unit 22 may use the Adam optimizer to train the discovery model 110 in a direction in which a mean squared error loss between the regression prediction value 121 and the actual data decreases.

As described above, the learning unit 22 may store the trained discovery model 110 in the database 10.

In the above-described learning system 20, the functions of the preprocessing unit 21 and the learning unit 22 may be performed by a processor implemented with one or more central processing units (CPU) or other chipsets, microprocessors, etc.

The prediction system 30 may use the discovery model 110 generated by the learning system 20 to predict candidate materials for treatment of the target protein for obtaining the candidate materials for treatment.

The prediction system 30 may include a preprocessing unit 31 and a prediction unit 32.

When the feature data of the target protein for discovery of the therapeutic candidate materials and the feature data of the candidate materials to verify whether the candidate materials are the candidate materials for treatment of the corresponding target protein are input, the preprocessing unit 31 may perform the preprocessing process of converting each of the feature data into graph data (G=(X, A)).

The prediction unit 32 may input the feature data of the target protein and the feature data of the candidate materials converted into the graph data by the preprocessing unit 31 to the discovery model, and determine whether the candidate materials are the candidate materials for treatment of the target protein based on the classification prediction value output from the discovery model. Referring to FIG. 2, the prediction unit 32 may input the graph data of the target protein and the graph data of the candidate materials to the 5-layer GINs 111 and 112 of the discovery model 110, respectively, and determine whether the candidate materials are the candidate materials for treatment of the target protein based on the classification prediction value 122 output from the classifier 116. In this case, the graph data of the candidate materials may be input as the input of the 5-layer GIN 112 instead of the graph data of the antibody used during the training.

In the above-described prediction system 30, the functions of the preprocessing unit 31 and the prediction unit 32 may be performed by a processor implemented with one or more CPUs, other chipsets, microprocessors, etc.

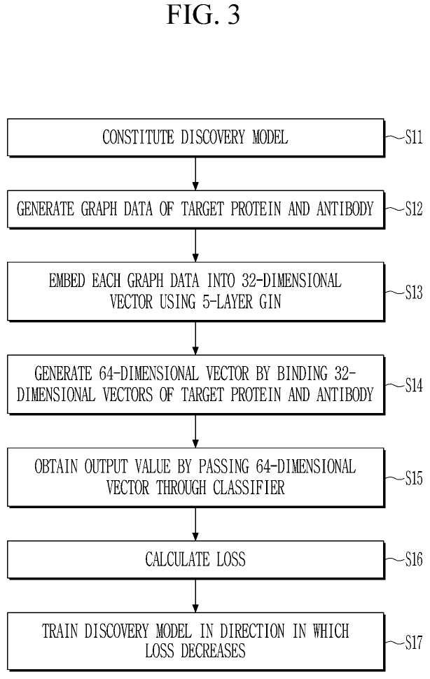

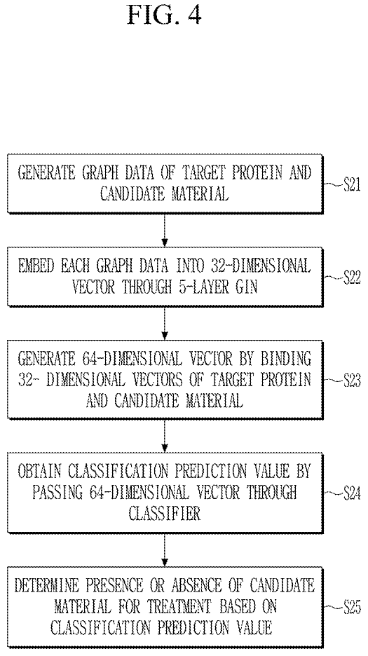

Hereinafter, referring to FIGS. 3 and 4, a method for discovering candidate materials of a target protein through the above-described system 1 for discovering candidate materials for treatment will be described in detail.

FIG. 3 schematically illustrates a method for generating a discovery model of the system 1 for discovering candidate materials for treatment according to an embodiment. The method for generating a discovery model of FIG. 3 may be performed by the learning system 20 described with reference to FIG. 1.

Referring to FIGS. 2 and 3, the learning system 20 may configure the discovery model 110 to include the two 5-layer GINs 111 and 112 and the classifier 116 (S11).

Thereafter, the learning system 20 reads the training data set composed of the feature data of the target protein and the corresponding antibody from the database 10, and generates the graph data of the target protein and antibody through the preprocessing of the read training data set, respectively (S12).

When the graph data is obtained through step S12, the learning system 20 may input the obtained graph data to the 5-layer GINs 111 and 112 of the discovery model 110, respectively, and use the 5-layer GINs 111 and 112 to embed each graph data into the 32-dimensional vectors 113 and 114 (S13).

The learning system 20 may generate the 64-dimensional vector 115 by combining the 32-dimensional vectors 113 and 114 of the target protein and antibody obtained through step S13 (S14), and obtain output values by passing the 64-dimensional vector 115 thus generated through the classifier 116 (S15). That is, the 64-dimensional vector 115 generated through step S14 may pass through the classifier 116 to obtain the regression prediction value 121 and the classification prediction value 122 (S15).

In step S15, the learning system 20 may adjust the output value of the two-layer MLP of the classifier 116 to the value between 0 and 1 using the sigmoid function, and then compare this value with a threshold (e.g., 0.5) to generate the classification prediction value 122.

In step S15, the classifier 116 of the learning system 20 may use the output value of the 2-layer MLP as the IC50 value to generate the regression prediction value 121 corresponding to the binding force of the target protein and antibody used.

When the output value is obtained from the classifier 116, the learning system 20 may calculate the loss by substituting the output value and the label of the data into the loss function (S16). The discovery model 110 may be trained in the direction in which the loss calculated through step S16 decreases (S17).

In step S17, the learning system 20 may use the Adam optimizer to train the discovery model 110 in the direction in which the loss of the binary cross-entropy loss between the classification prediction value 122 output from the discovery model 110 and the actual data decreases. In addition, the learning system 20 may use the Adam optimizer to train the discovery model 110 in the direction in which the mean squared error loss decreases.

When the discovery model 110 is obtained by the training through steps S11 to S17 described above, the learning system 20 may store the discovery model 110 in the database 10 so that the discovery model 110 may be used in the prediction system 30.

FIG. 4 schematically illustrates a method for discovering candidate materials of the system 1 for discovering candidate materials for treatment according to an embodiment. The method for discovering candidate materials of FIG. 4 may be performed by the prediction system 30 described with reference to FIG. 1.

Referring to FIGS. 2 and 4, when the feature data of the target protein for discovery of the therapeutic candidate materials and the feature data of the feature data of the candidate materials to verify whether the therapeutic candidate materials are the candidate materials for treatment of the target protein are input, the prediction system 30 may generate the graph data of the target protein and the candidate material, respectively, from the feature data through the preprocessing (S21).

When the graph data is obtained through step S21, the prediction system 30 may input the obtained graph data to the 5-layer GINs 111 and 112 of the discovery model 110, respectively, and use the 5-layer GINs 111 and 112 to embed each graph data into the 32-dimensional vectors 113 and 114 (S22).

Thereafter, the prediction system 30 may generate the 64-dimensional vector 115 by combining the 32-dimensional vectors 113 and 114 of the target protein and antibody obtained through step S22 (S23), and obtain the classification prediction value by passing the 64-dimensional vector 115 thus generated through the classifier 116 (S24).

In addition, the prediction system 30 may finally determine whether the candidate materials used as the input of the discovery model 110 are the candidate materials for treatment of the target protein based on the classification prediction value output from the classifier 116 of the discovery model 110 (S25).

Hereinafter, the effects of the above-described system 1 for discovering candidate materials for treatment will be described with reference to Experimental Example.

### Experimental Example

The experiment of the system 1 for discovering candidate materials for treatment was based on the discovery model 110 of FIG. 2 described above, but performed experiment by a method for performing the training at a learning rate of 0.0001 by 1000 epochs and then measuring performance using a verification data set.

### Comparative Example

VirusNet's tree-based model was used as Comparative Example. The VirusNet's tree-based model is a model that predicts the binding of the antibody to the target protein without using the structural characteristics of the graph data of the target protein and antibody for the training.

Table 1 below compares the prediction performance of the above-described Experimental example and Comparative Example.

TABLE 1

| | Comparative | Experimental |
|---|---|---|
| Output Value | Example | Example |
| Classification | 76.31% | 97.00% |
| (Performance measurement: Accuracy) | | |
| Regression | 6.36 | 4.85 |
| (Performance measurement: RMSE) | | |

Referring to Table 1 above, the classification accuracy of the discovery model 110 according to the embodiment was 97.00%, which showed improved classification accuracy by 20.69% compared to VirusNet's model. In addition, a root mean square error (RMSE) of the regression output value of the discovery model 110 was 4.85, and decreased by 1.51 compared to the VirusNet's model. In this way, the system 1 for discovering candidate materials for treatment according to the embodiment may improve the discovery performance of the therapeutic candidate materials by configuring the discovery model 110 based on the GIN, which may well express the structural features of the graph.

Embodiments of the present disclosure described above are not implemented through only the apparatus and/or the method described above, but may also be implemented through a program executing functions corresponding to configurations of embodiments of the present disclosure or a recording medium in which the program is recorded. In addition, this implementation may be easily made by those skilled in the art to which the present disclosure pertains from embodiments described above.

Although the embodiment of the present disclosure has been described in detail hereinabove, the scope of the present disclosure is not limited thereto. That is, several modifications and alterations made by a person of ordinary skill in the art using a basic concept of the present disclosure as defined in the claims fall within the scope of the present disclosure.

1. A system for discovering candidate materials for treatment, comprising:
a prediction system that inputs first graph data of a target protein and second graph data of the candidate materials to a prediction model and determines whether the candidate materials are candidate materials for treatment of the target protein based on an output value output from the prediction model in response to the first and second graph data,
wherein the prediction model is a graph neural networks (GNN)-based model for predicting presence or absence of binding between the target protein and the candidate material.

2. The system of claim 1, wherein:
the prediction model includes:
a first multi-layer graph isomorphism network (GIN) that embeds input graph data into a first vector;
a second multi-layer GIN that embeds the input graph data into a second vector; and
a classifier that generates the output value by passing a third vector generated by combining the first vector and the second vector through a multi-layer perceptron (MLP), and
the first and second graph data are input to the first and second multi-layer GINs, respectively.

3. The system of claim 2, wherein:
the first and second multi-layer GINs are each composed of a 5-layer GIN.

4. The system of claim 2, wherein:
the first vector and the second vector are each a 32-dimensional vector, and the third vector is a 64-dimensional vector.

5. The system of claim 2, wherein:
the MLP is a two-layer MLP.

6. The system of claim 1, wherein:
the prediction system converts feature data of the target protein and the candidate materials into the first graph data and the second graph data, respectively, and
the feature data is amino acid sequence data for a binding site.

7. The system of claim 1, further comprising:
a learning system that trains the prediction model using a plurality of training data sets,
wherein the plurality of training data sets include a plurality of target proteins and feature data for antibodies of each of the plurality of target proteins, and
the learning system converts feature data of the target protein and antibody corresponding to each other into third graph data and fourth graph data, respectively, and trains the prediction model using the third graph data and the fourth graph data.

8. The system of claim 7, wherein:
the learning system calculates a loss using the output value output from the prediction model and a loss function after the third graph data and the fourth graph data are input to the prediction model, and trains the prediction model in a direction in which the loss is minimized.

9. A method for discovering candidate materials for treatment in a system for discovering candidate materials for treatment, the method comprising:
inputting first graph data of a target protein and second graph data of the candidate materials to a prediction model to obtain a prediction value for presence or absence of binding between the target protein and the candidate material; and
determining whether the candidate materials are candidate materials for treatment of the target protein based on the prediction value,
wherein the prediction model is a GNN-based model for predicting presence or absence of binding between the target protein and the candidate material.

10. The method of claim 9, wherein:
the acquiring of the prediction value includes:
embedding the first and second graph data into first and second vectors, respectively, through first and second multi-layer GINs constituting the prediction model; and
acquiring the prediction value by passing a third vector generated by combining the first and second vectors through an MLP of a classifier constituting the prediction model.

11. The method of claim 10, wherein:
the first and second multi-layer GINs are each composed of a 5-layer GIN, and
the first vector and the second vector are each a 32-dimensional vector, and the third vector is a 64-dimensional vector.

12. The method of claim 9, further comprising:
converting feature data of the target protein and the candidate materials into the first graph data and the second graph data, respectively,
wherein the feature data is amino acid sequence data for a binding site.

13. The method of claim 9, further comprising:
converting feature data of the target protein and antibody corresponding to each other into third graph data and fourth graph data, respectively; and
training the prediction model using the third graph data and the fourth graph data.

14. The method of claim 13, wherein:
the training includes:
embedding the third and fourth graph data into fourth and fifth vectors, respectively, through first and second multi-layer GINs constituting the prediction model;
acquiring a prediction value by passing a sixth vector generated by combining the fourth and fifth vectors through the MLP of the classifier constituting the prediction model;
calculating a loss using the prediction value output from the prediction model and a loss function; and
training the prediction model in a direction in which the loss is minimized.

15. The method of claim 14, wherein:
the prediction value output from the prediction model includes a classification prediction value for the presence or absence of binding between the target protein and antibody corresponding to the third and fourth graph data, and
the training of the prediction model in the direction in which the loss is minimized includes
training the prediction model in a direction in which a binary cross-entropy loss between the classification prediction value and actual data decreases using an Adam optimizer.

16. The method of claim 15, wherein:
the prediction value output from the prediction model includes a regression prediction value for binding force of the target protein and antibody corresponding to the third and fourth graph data, and
the training of the prediction model in the direction in which the loss is minimized includes
training the prediction model in a direction in which a mean squared error loss between the regression prediction value and actual data decreases using an Adam optimizer.
